(19) United States
(12) Patent Application Publication
(10) Pub. No.: US 2013/0068212 A1
(43) Pub. Date: Mar. 21, 2013

(54) Pocket warmer able to give out odors

(71) Applicants: Ming-Hui Chen, Tainan City (TW); Ling-Ling Huang, Taipei City (TW)

(72) Inventors: Ming-Hui Chen, Tainan City (TW); Ling-Ling Huang, Taipei City (TW)

(21) Appl. No.: 13/200,172

(22) Filed: Sep. 21, 2011

(51) Int. Cl. A61F 7/08; A61L 9/03

(52) U.S. Cl. 126/204

(57) ABSTRACT

A pocket warmer able to give out odors is provided with a combustion unit and a placing stand assembled over the combustion unit for receiving aromatic substance or essential oil with an odor a user wants. Thus, when the combustion unit of the pocket warmer is heated, hot air produced will rise and carry out heating to the aromatic substance or the essential oil of the placing stand over the combustion unit, able to attain effects of keeping warm and giving out odors, and safe and practical in use.

### BACKGROUND OF THE INVENTION

1. Field of the Invention

This invention relates to a pocket warmer able to give out odors when the pocket warmer is heated.

2. Description of the Prior Art

Generally, most conventional pocket warmers have only one single function of heating and preserving heat so the conventional pocket warmers are used mostly in winter only. In this case, manufacturers of pocket warmers seldom receive procurement lists of pocket warmers from their customers in other seasons, In view of this situation, a pocket warmer, which has essential oil or insect powder directly filled into the pocket warmer to be burnt for giving out odors, has been developed and used; nevertheless, direct burning of the essential oil or the vermifuge may produce harmful odors.

### SUMMARY OF THE INVENTION

The objective of this invention is to offer a pocket warmer able to give out odors and keep warm when the pocket warmer is heated for use.

The pocket warmer in the present invention includes a main body formed with a fitting protruding edge. An upper casing is covered around the fitting protruding edge of the main body and provided with a fitting portion and a plurality of air holes. A combustion unit combined on the topside of the main body and disposed with an engage edge and heat-dissipating holes. A placing stand to be assembled over the combustion unit is mounted thereon with a fitting holder with a support surface, and a horizontal placing surface. Aromatic substance is placed on the support surface of the placing stand.

### BRIEF DESCRIPTION OF DRAWINGS

This invention will be better understood by referring to the accompanying drawings, wherein:

FIG. 1 is an exploded perspective view of a first preferred embodiment of a pocket warmer able to give out odors in the present invention;

FIG. 2 is a partial perspective view of the first preferred embodiment of the pocket warmer able to give out odors in the present invention;

FIG. 3 is a partial cross-sectional view of the first preferred embodiment of the pocket warmer able to give out odors in the present invention;

FIG. 4 is an exploded perspective view of a second preferred embodiment of a pocket warmer able to give out odors in the present invention;

FIG. 5 is a partial perspective view of the second preferred embodiment of the pocket warmer able to give out odors in the present invention;

FIG. 6 is a partial perspective view of the second preferred embodiment of the pocket warmer able to give out odors in the present invention;

FIG. 7 is a perspective view of a third preferred embodiment of a pocket warmer able to give out odors in the present invention; and

FIG. 8 is an exploded perspective view of a display stand for the pocket warmer in the present invention.

### DETAILED DESCRIPTION OF THE PREFERRED EMBODIMENT

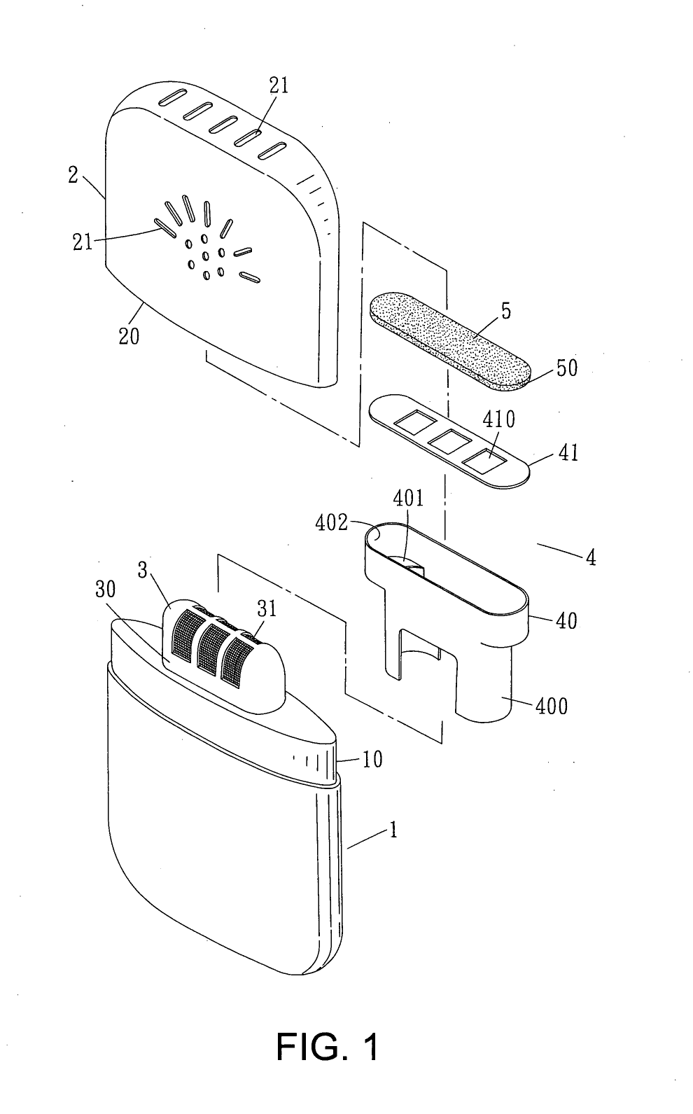

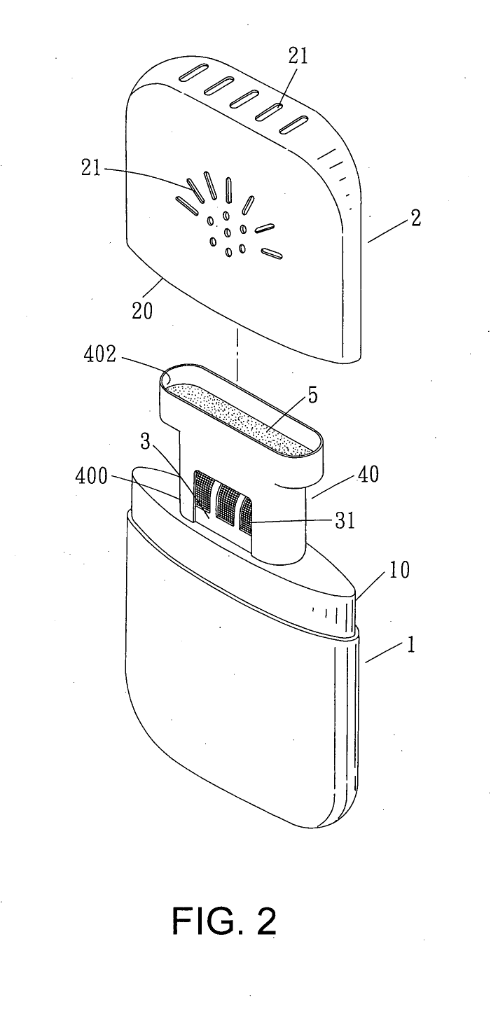

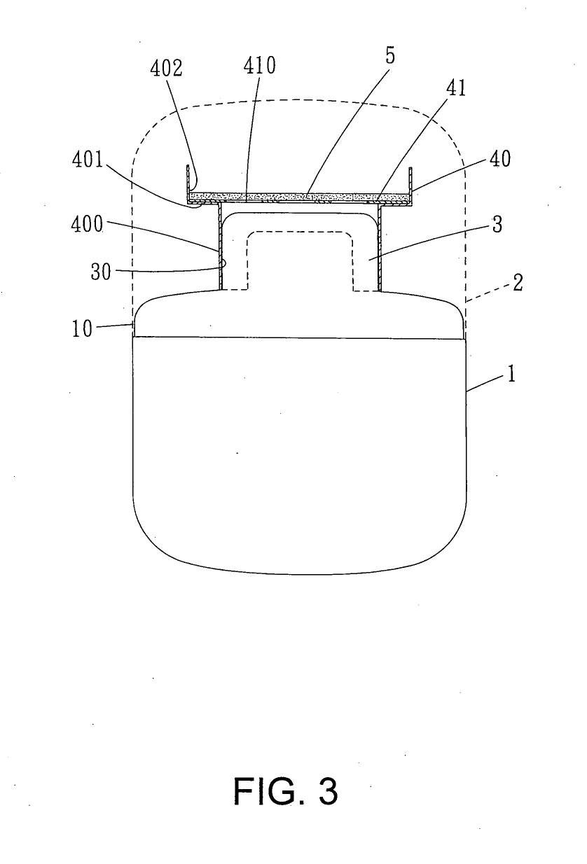

A first preferred embodiment of a pocket warmer able to give out odors in the present invention, as shown in FIGS. 1-3, includes a main body 1, an upper casing 2, a combustion unit 3, and a placing stand 4 as main components combined together.

The main body 1 has an upper portion formed with a fitting protruding edge 10.

The upper casing 2 to be covered around the fitting protruding edge 10 of the main body 1 is hollow, provided with a fitting portion 20 and a plurality of air holes 21.

The combustion unit 3 is combined on the topside of the main body 1, having its outer edge disposed with an engage edge 30 and its topside bored with plural heat-dissipating holes 31.

The placing stand 4 is provided with a hollow fitting holder 40 and a horizontal placing surface 41. The fitting holder 40 is of an oval shape, having a hollow space and its lower side formed with an engage portion 400 and its upper side provided with a support surface 401 with an oval and center-hollowed depth, and an engage inner wall 402. The engage portion 400 of the hollow fitting holder 40 fit tight with the engage edge 30 of the combustion unit 3, and the horizontal placing surface 41 with plural through holes 410 is fixed on the support surface 401.

An aromatic substance 5 is placed on the horizontal placing surface 41 of the placing stand 4, and the peripheral edge 50 of the aromatic substance 5 is slightly larger than the engage inner wall 402 of the hollow fitting holder 40. The aromatic substance 5 can be aromatic components, insect repelling powder plate, or essential oil.

To use the pocket warmer outdoors, firstly, remove the upper casing 2 from the main body 1 and ignite the combustion unit 3 at the topside of the main body 1. Next, the horizontal placing surface 41 is positioned on the support surface 401 and the aromatic substance 5 containing insect repelling powder is fixedly inlaid with the engage inner wall 402 of the hollow fitting holder 40, and then the upper casing 2 has its fitting portion 20 combined with the fitting protruding edge 10 of the main body 1. At this time, the combustion unit 3 at the topside of the main body 1 will give out hot air and simultaneously, the hot sir will rise through the hollow support surface 401 of the fitting holder 40 and go through the through holes 410 of the horizontal placing surface 41 to heat the aromatic substance 5 for producing evaporative odors to be diffused outward through the air holes 21 of the upper casing 2. Thus, the pocket warmer can be placed on a table or put beside a user for expelling worms and mosquitoes.

To use the pocket warmer indoors, the aromatic substance 5 containing fragrant ingredients is placed on the horizontal placing surface 41 of the placing stand 4 to enable the pocket warmer to give out fascinating fragrance.

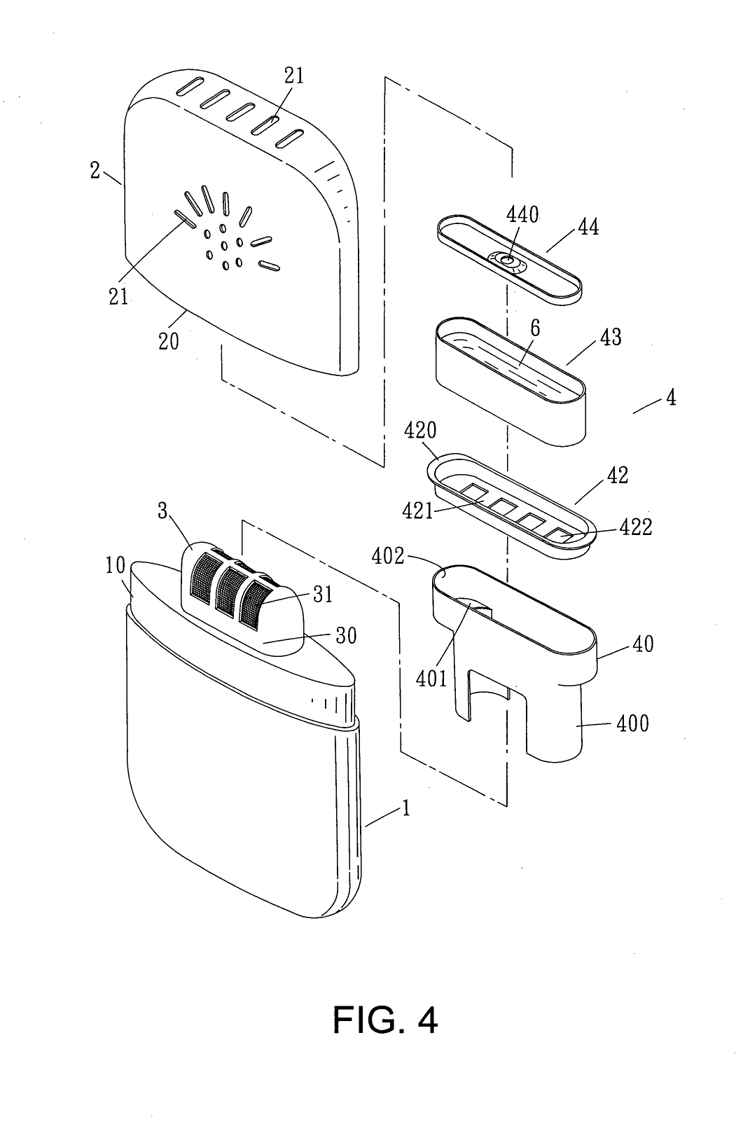

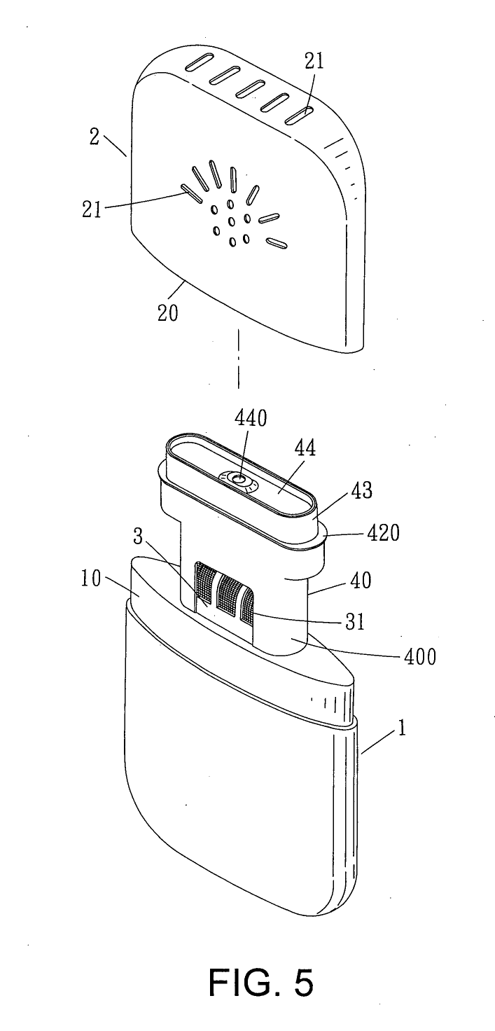

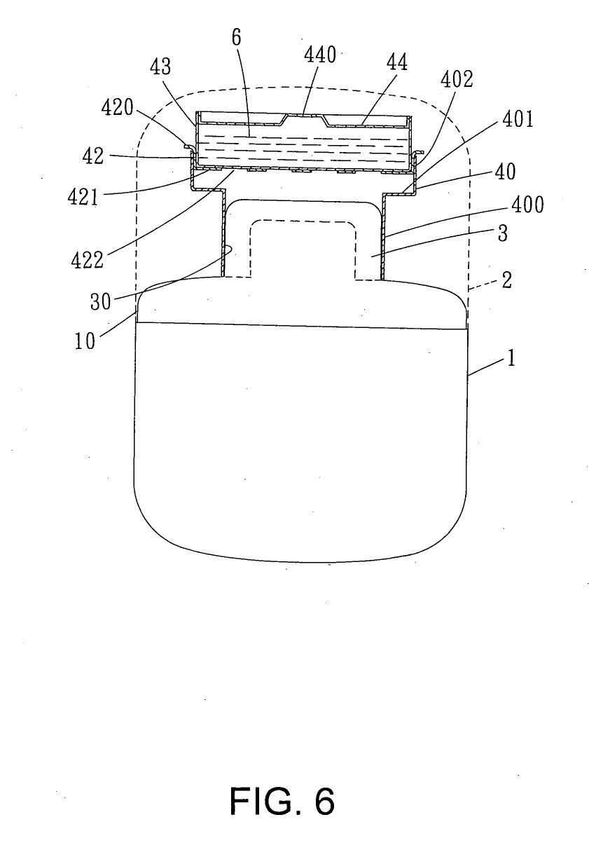

A second preferred embodiment of a pocket warmer able to give out odors in the present invention, as shown in FIGS. 4-6, includes a main body 1, an upper casing 2, a combustion unit 3, and a placing stand 4 as main components combined together.

The main body 1 has its upper section formed with a fitting protruding edge 10; the upper casing 2 to be covered around the fitting protruding edge 10 of the main body 1 is hollow, provided with a fitting portion 20 and a plurality of air holes 21, and the combustion unit 3 is secured on the topside of the main body 1, having its outer edge disposed with an engage edge 30 and its topside bored with plural heat-dissipating holes 31. The placing stand 4 is composed of a hollow fitting holder 40, a placing plate 42, an oil box 43 and an oil box cover 44. The fitting holder 40 is oval shape, having an empty space and its lower side provided with an engage portion 400 and its upper side disposed with a support surface 401 with an oval and center-hollowed depth, and an engage inner wall 402. The engage portion 400 of the fitting holder 40 and the engage edge 30 of the combustion unit 3 fit tightly together, and the placing plate 42 is mounted on the fitting holder 40, having two sides respectively formed with a lug 420 and its bottom provided with a placing surface 421 bored with a plurality of air holes 422. The oil box 43 is placed on the placing surface 421 of the placing plate 42, and the oil box cover 44 is covered on the oil box 43, bored with an oil-filling air opening 440, and essential oil 6 is stored in the oil box 43 of the placing stand 4.

To use the pocket warmer outdoors, firstly, remove the upper casing 2 from the main body 1 and ignite the combustion unit 3 at the topside of the main body 1 and, after the combustion unit 3 is in a smoldering state, the oil box 43 filled with the essential oil 6 is positioned in the placing plate 42, which is then firmly engaged with and positioned on the fitting holder 40. Subsequently, the upper casing 2 has its fitting portion 20 combined with the fitting protruding edge 10 of the main body 1. At this time, the combustion unit 3 at the topside of the main body 1 will give out hot air, and simultaneously the hot air will rise from the support surface 401 of the fitting holder 40 and then go through the air holes 422 of the placing plate 42 to heat the essential oil 6 in the oil box 43 for giving out fragrant odor, which will immediately be diffused outward through the air holes 21 of the upper casing 2. Thus, the pocket warmer 1 can be placed on a table or put beside a user for giving out fascinating fragrance.

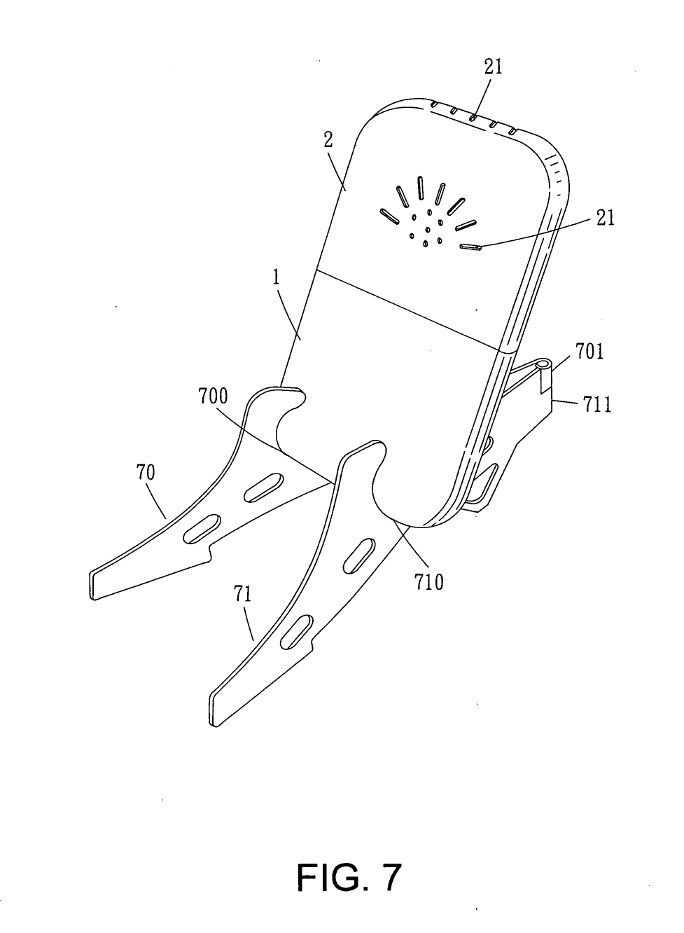

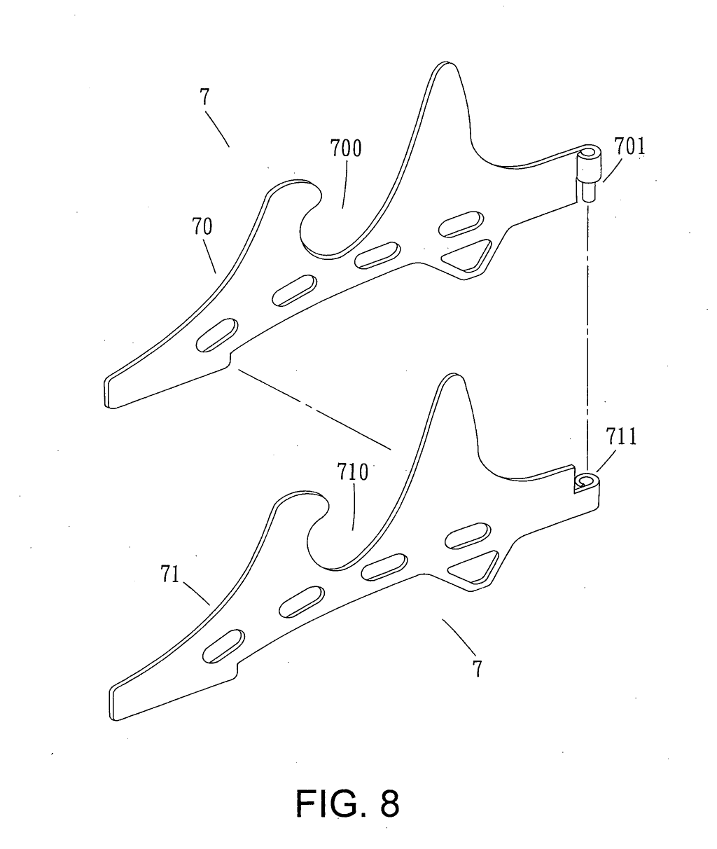

A third preferred embodiment of a pocket warmer able to give out odors in the present invention, as shown in FIGS. 7 and 8, is composed of a main body 1, an upper casing 2, a combustion unit 3, a placing stand 4, which are all the same components as those of the first preferred embodiment, and a display stand 7 added. Then the aromatic substance 5 is placed in the hollow fitting holder 40 on the horizontal placing surface 41. The display stand 7 is made by pivotally connecting two support frames 70 and 71 together, which are respectively provided with a placing groove 700, 710 and a pivotal portion 701, 711.

When the pocket warmer of the third preferred embodiment is used outdoors or indoors, the aromatic substance 5 containing insect repelling powder is placed on the placing stand 4 to be heated to produce evaporative odors when the combustion unit 3 of the main body 1 is ignited. Afterward, stretch outward the two support frames 70 and 71 of the display stand 7 to form an included angle and put the main body 1 of the pocket warmer in the placing grooves 700 and 710 and then have the pocket warmer together with the display stand 7 placed on a table or put beside a user, able to attain effects of expelling worms and preventing mosquitoes.

As can be understood from the above description, this invention has the following advantages.

1. The pocket warmer of this invention carries out heating in a way of smoldering, safe in use.

2. The pocket warmer of this invention can be used not only for giving out fragrance but also for expelling mosquitoes, convenient and practical in use.

3. The pocket warmer of this invention carries out heating to the aromatic substance or to the insect powder with hot air, impossible to produce harmful odors.

While the preferred embodiments of the invention have been described above, it will be recognized and understood that various modifications may be made therein and the appended claims are intended to cover all such modifications that may fall within the spirit and scope of the invention.

What is claimed is:

1. A pocket warmer able to give out odors comprising:
a main body formed with a fitting protruding edge;
an upper casing covered around said fitting protruding edge of said main body, said upper casing provided with a fitting portion and plural air holes;
a combustion unit fixed on a topside of said main body, said combustion unit disposed with an engage edge and plural heat-dissipating holes;
a placing stand assembled over said combustion unit, said placing stand mounted thereon with a fitting holder and a horizontal placing surface, said fitting holder formed with a support surface;
a aromatic substance positioned on said support surface of said placing stand; and
Foresaid components combined together to make up said pocket warmer that has function of keeping warm and giving out odors.

2. A pocket warmer able to give out odors as claimed in claim 1, wherein said aromatic substance is insect repelling powder plate.

3. A pocket warmer able to give out odors as claimed in claim 1, wherein said aromatic substance is essential oil.

4. A pocket warmer able to give out odors as claimed in claim 1, wherein a display stand is provided for said pocket warmer to be laid thereon.

5. A pocket warmer able to give out odors comprising:
a main body formed with a fitting protruding edge;
an upper casing covered around said fitting protruding edge of said main body, said upper casing provided with a fitting portion and a plurality of air holes;
a combustion unit secured on a topside of said main body, said combustion unit disposed with an engage edge and a plurality of heat-dissipating holes;
a placing stand mounted over said combustion unit, said placing stand composed of a fitting holder, a placing plate, an oil box, an oil box cover, and an oil box cover covered on said oil box and bored with an oil-filling air opening; and
essential oil deposited in said oil box of said placing stand.

6. A pocket warmer able to give out odors as claimed in claim 5, wherein a display stand is provided for said pocket warmer to be laid thereon.
